(19) United States
(12) Patent Application Publication
(10) Pub. No.: US 2021/0267663 A1
(43) Pub. Date: Sep. 2, 2021

(54) ELECTROSURGICAL FORCEPS INCLUDING SENSOR FEEDBACK FACILITATING TISSUE SEALING AND/OR DETERMINATION OF A COMPLETED SEAL

(71) Applicant: Covidien LP, Mansfield, MA (US)

(72) Inventors: Amanda H. Lennartz, Erie, CO (US); Daniel A. Joseph, Golden, CO (US); Jennifer R. McHenry, Denver, CO (US); Cornelia F. Twomey, Longmont, CO (US); Erin E. Wehrly, Longmont, CO (US); Pierre Gherardi, Longmont, CO (US); Tracy J. Pheneger, Longmont, CO (US); Tyler J. Bagrosky, Arvada, CO (US); Robert H. Wham, Boulder, CO (US); Jing Zhao, Superior, CO (US)

(21) Appl. No.: 17/181,298

(22) Filed: Feb. 22, 2021

Related U.S. Application Data
(60) Provisional application No. 62/984,066, filed on Mar. 2, 2020.

(51) Int. Cl. A61B 18/14; A61B 34/30

(52) U.S. Cl. CPC A61B 18/1445; A61B 2017/00022; A61B 34/30

(57) ABSTRACT

An electrosurgical system includes an end effector assembly and an electrosurgical generator. The end effector assembly includes first and second jaw members, one or both of which is movable relative to the other for grasping tissue between tissue-contacting surfaces thereof. One or both of the jaw members includes a sensor configured to sense at least one property associated with the grasped tissue. The electrosurgical generator includes a controller and an energy output configured to supply electrosurgical energy to the tissue-contacting surface of one or both of the jaw members for conduction through the grasped tissue to seal the grasped tissue. The controller is configured to receive the at least one sensed property, determine at least one condition of collagen within the grasped tissue based upon the at least one sensed property, and control the energy output based upon the determined at least one condition of the collagen within the grasped tissue.

### CROSS-REFERENCE TO RELATED APPLICATIONS

This application claims the benefit of, and priority to, U.S. Provisional Patent Application No. 62/984,066, filed on Mar. 2, 2020, the entire contents of which are hereby incorporated herein by reference.

### FIELD

The present disclosure relates to electrosurgical instruments and, more particularly, to electrosurgical forceps including sensors providing feedback to facilitate tissue sealing and/or determination of a completed seal.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to seal tissue. Typically, once tissue is sealed, the surgeon has to accurately sever the sealed tissue. Accordingly, many electrosurgical forceps are designed to incorporate a knife or cutting member utilized to effectively sever the sealed tissue.

### SUMMARY

As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an electrosurgical system including an end effector assembly and an electrosurgical generator. The end effector assembly includes first and second jaw members each defining an electrically-conductive tissue-contacting surface. One or both of the jaw members is movable relative to the other between a spaced-apart position and an approximated position for grasping tissue between the tissue-contacting surfaces thereof. One or both of the jaw members includes a sensor configured to sense at least one property associated with the grasped tissue. The electrosurgical generator includes a controller and an energy output. The energy output is configured to supply electrosurgical energy to the tissue-contacting surface of at least one of the first or second jaw members for conduction through the grasped tissue to seal the grasped tissue. The controller is configured to receive the at least one sensed property, determine at least one condition of collagen within the grasped tissue based upon the at least one sensed property, and control the energy output based upon the determined at least one condition of the collagen within the grasped tissue.

In an aspect of the present disclosure, the at least one condition of the collagen includes: denaturation of the collagen, migration of fibers of the collagen, restructuring of the collagen, crosslinking of the collagen, a type of the crosslinking of the collagen, a phase of the collagen, or a phase-change of the collagen.

In another aspect of the present disclosure, the sensor includes an electrical sensor and the controller includes a machine learning algorithm configured to determine the at least one condition of the collagen based upon the at least one sensed property received from the electrical sensor.

In another aspect of the present disclosure, the sensor includes at least one of: an optical sensor, an electrical sensor, a mechanical property sensor, or a chemical sensor.

In still another aspect of the present disclosure, the controller is further configured to determine whether the grasped tissue is sufficiently sealed based upon the determined at least one condition of the collagen.

In yet another aspect of the present disclosure, controlling the energy output includes at least one of: starting, modifying, continuing, or stopping the energy supplied to the at least one tissue-contacting surface.

In still yet another aspect of the present disclosure, the controller includes a storage device storing a machine learning algorithm configured to determine the at least one condition of collagen based upon the at least one sensed property.

In another aspect of the present disclosure, a housing and a shaft extending distally from the housing are provided. The end effector assembly is disposed at a distal end portion of the shaft in such aspects. A manual actuator, e.g., handle, may be coupled to the housing and configured to move the at least one of the first or second jaw members between the spaced-apart position and the approximated position.

In another aspect of the present disclosure, first and second shaft members pivotably coupled to one another about a pivot are provided. In such aspects, the end effector assembly extends distally from the pivot and the first and second shaft members are movable relative to one another to move the at least one of the first or second jaw members between the spaced-apart position and the approximated position.

In yet another aspect of the present disclosure, a robotic arm is provided wherein the end effector assembly extends distally from the robotic arm.

A method of sealing tissue in accordance with the present disclosure includes grasping tissue between electrically-conductive tissue-contacting surfaces of first and second jaw members, supplying electrosurgical energy to the tissue-contacting surface of at least one of the first or second jaw members for conduction through the grasped tissue, sensing at least one property associated with the grasped tissue, determine at least one condition of collagen within the grasped tissue based upon the at least one sensed property, and controlling the supplying of electrosurgical energy based upon the determined at least one condition of the collagen within the grasped tissue.

In an aspect of the present disclosure, the at least one condition of the collagen includes: denaturation of the collagen, migration of fibers of the collagen, restructuring of the collagen, crosslinking of the collagen, a type of the crosslinking of the collagen, a phase of the collagen, or a phase-change of the collagen.

In another aspect of the present disclosure, the at least one property is sensed by an electrical sensor and determining the at least one condition includes implementing a machine learning algorithm to determine the at least one condition based upon the at least one sensed property sensed by the electrical sensor.

In another aspect of the present disclosure, the sensed at least one property is an optical property, an electrical property, a mechanical property, or a chemical property.

In still another aspect of the present disclosure, the method further includes determining whether the grasped tissue is sufficiently sealed based upon the determined at least one condition of the collagen.

In yet another aspect of the present disclosure, controlling the supplying of electrosurgical includes at least one of: starting, modifying, continuing, or stopping the supply of energy.

In still yet another aspect of the present disclosure, determining the at least one condition of collagen includes running a machine learning algorithm to determine the at least one condition of collagen based upon the at least one sensed property.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure connected to an electrosurgical generator;

FIG. 2A is a perspective view of a distal end portion of the forceps of FIG. 1, wherein jaw members of an end effector assembly of the forceps are disposed in a spaced-apart position;

FIG. 2B is a perspective view of the distal end portion of the forceps of FIG. 1, wherein the jaw members are disposed in an approximated position;

FIG. 3 is a perspective view of a hemostat-style electrosurgical forceps provided in accordance with the present disclosure;

FIG. 4 is a schematic illustration of a robotic surgical instrument provided in accordance with the present disclosure;

FIG. 5 is a block diagram of the electrosurgical generator of FIG. 1;

FIG. 6 is a block diagram of a controller of the electrosurgical generator of FIG. 5;

FIG. 7 is a logic diagram of a machine learning algorithm in accordance with the present disclosure; and

FIGS. 8A-8D are transverse, cross-sectional views of the jaw members of the end effector assembly of FIG. 2A shown grasping tissue therebetween and including various different sensor mechanisms incorporated into one or both of the jaw members.

### DETAILED DESCRIPTION

The present disclosure provides electrosurgical instruments including sensor feedback to facilitate tissue sealing and/or determination of a completed tissue seal. Tissue sealing is defined as the process of denaturing and liquefying the collagen in tissue so that it crosslinks and reforms into a fused mass. The present disclosure, more specifically, provides sensor feedback to determine, in real-time (allowing computer processing time within a suitable real-time constraint), a state, property, and/or other condition of the collagen in tissue before, during, and/or after the application of energy to the tissue, thus facilitating tissue sealing by enabling the application of energy to start, continue, change, or stop based upon the sensor feedback. The state, property, and/or other condition of the collagen in the tissue is additionally or alternatively used to facilitate determination, in real-time, of whether tissue has been sufficiently sealed. The state, property, and/or other condition of the collagen may include: the presence and/or extent of denaturation of the collagen; the presence and/or extent of migration of collagen fibers; the presence, extent, and/or type of collagen restructuring; the presence, extent, and/or type (reducible or non-reducible) of reformed collagen crosslinks; a phase of the collagen; a phase-change of the collagen; etc.

Various exemplary electrosurgical instruments and sensor mechanisms are detailed below; however, the aspects and features of the present disclosure are not limited thereto as any other suitable electrosurgical instruments and/or sensor mechanisms are also contemplated for use in accordance with the present disclosure.

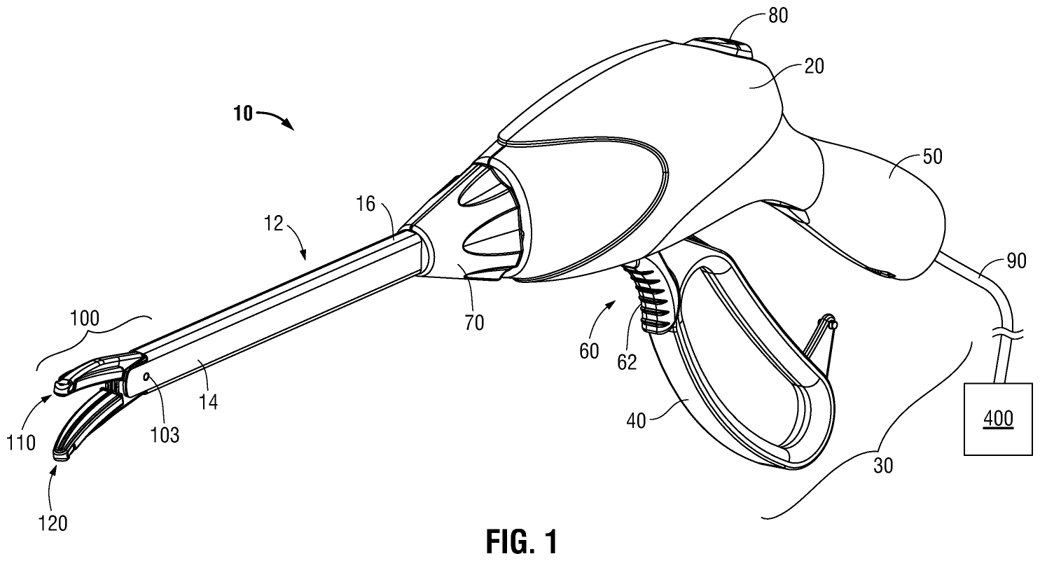

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

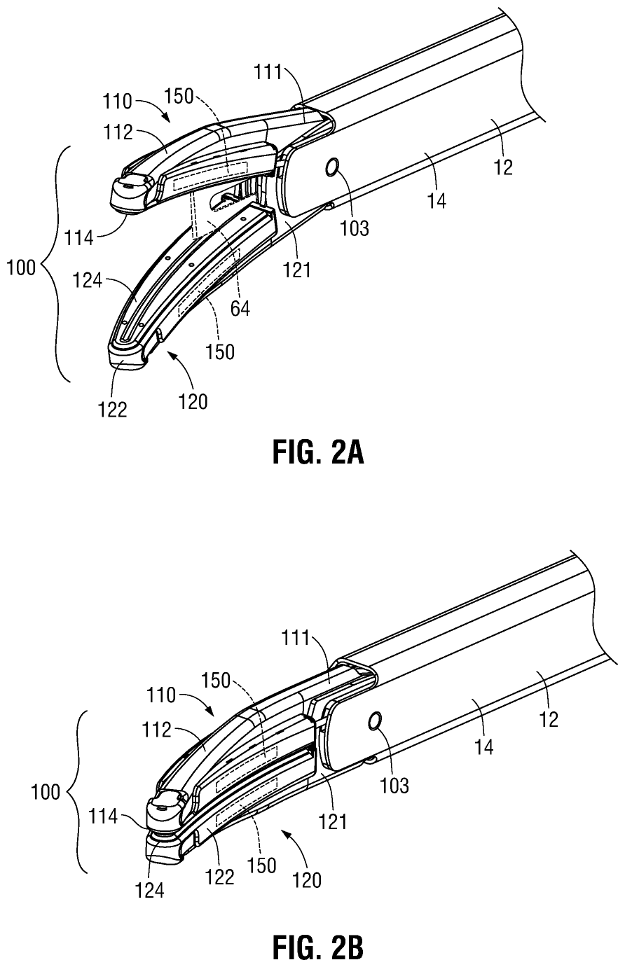

Forceps 10 includes a housing 20, a handle assembly 30, a trigger assembly 60, a rotating assembly 70, an activation switch 80, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable 90 that connects forceps 10 to an electrosurgical generator 400. Cable 90 includes a wire (or wires) (not shown) extending therethrough that has sufficient length to extend through shaft 12 in order to provide energy to one or both tissue-contacting surfaces 114, 124 of jaw members 110, 120, respectively, of end effector assembly 100 (see FIGS. 2A and 2B). Activation switch 80 is coupled to tissue-contacting surfaces 114, 124 (FIGS. 2A and 2B) and electrosurgical generator 400 for enabling the selective activation of the supply of energy to jaw members 110, 120 for sealing tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position (FIG. 2A) and an approximated position (FIG. 2B) to grasp tissue between jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120 (FIG. 2B).

Trigger assembly 60 includes a trigger 62 coupled to housing 20 and movable relative thereto between an un-actuated position and an actuated position. Trigger 62 is operably coupled to a knife 64 (FIG. 2A), so as to actuate knife 64 (FIG. 2A) to cut tissue grasped between jaw members 110, 120 of end effector assembly 100 upon actuation of trigger 62. As an alternative to knife 64, other suitable mechanical, electrical, or electromechanical cutting mechanisms (stationary or movable) are also contemplated.

With additional reference to FIGS. 2A and 2B, end effector assembly 100, as noted above, includes first and second jaw members 110, 120. Each jaw member 110, 120 includes a proximal flange portion 111, 121, an outer insulative jaw housing 112, 122 disposed about the distal portion (not explicitly shown) of each jaw member 110, 120, and a tissue-contacting surface 114, 124, respectively. Proximal flange portions 111, 121 are pivotably coupled to one another about pivot 103 for moving jaw members 110, 120 between the spaced-apart and approximated positions, although other suitable mechanisms for pivoting jaw members 110, 120 relative to one another are also contemplated. The distal portions (not explicitly shown) of the jaw members 110, 120 are configured to support jaw housings 112, 122, and tissue-contacting surfaces 114, 124, respectively, thereon.

Outer insulative jaw housings 112, 122 of jaw members 110, 120 support and retain tissue-contacting surfaces 114, 124 on respective jaw members 110, 120 in opposed relation relative to one another. Tissue-contacting surfaces 114, 124 are at least partially formed from an electrically conductive material, e.g., for conducting electrical energy therebetween for sealing tissue, although tissue-contacting surfaces 114, 124 may alternatively be configured to conduct any suitable energy, e.g., thermal, microwave, light, ultrasonic, etc., through tissue grasped therebetween for energy-based tissue sealing. As mentioned above, tissue-contacting surfaces 114, 124 are coupled to activation switch 80 and electrosurgical generator 400, e.g., via the wires (not shown) extending from cable 90 through forceps 10, such that energy may be selectively supplied to tissue-contacting surface 114 and/or tissue-contacting surface 124 and conducted therebetween and through tissue disposed between jaw members 110, 120 to seal tissue.

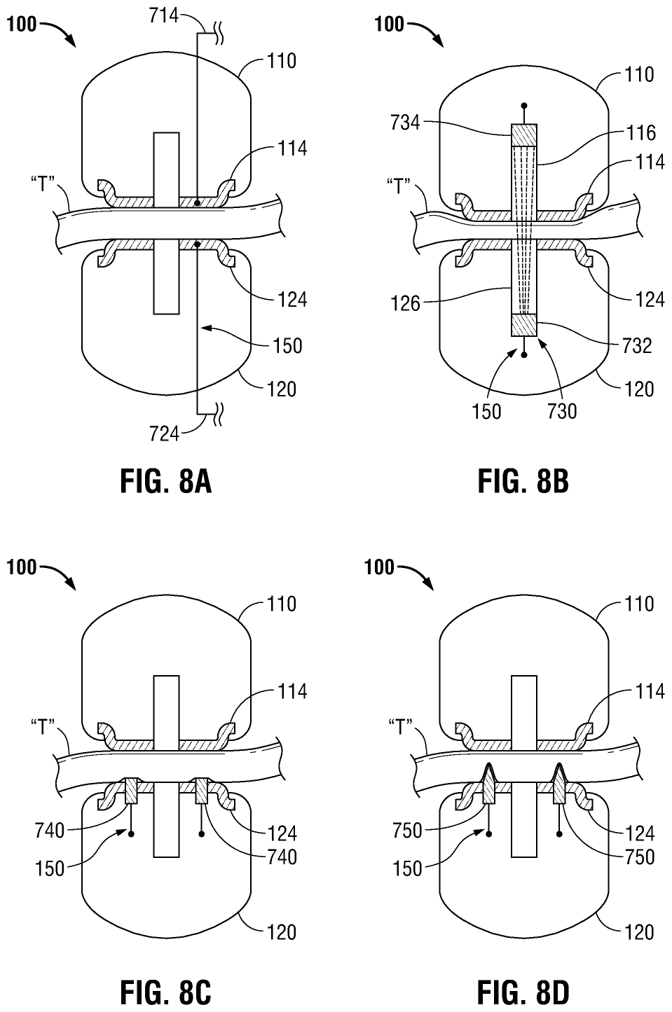

Continuing with reference to FIGS. 2A and 2B, end effector assembly 100 further includes a sensor mechanism 150 including components disposed within, on, or otherwise associated with one or both of jaw members 110, 120. Sensor mechanism 150 is configured to sense one or more properties (mechanical, optical, chemical, electrical, etc.) of tissue grasped between jaw members 110, 120 and to provide sensor feedback to generator 400 (FIG. 1) to enable determination of a state, property, and/or other condition of the collagen in tissue before, during, and/or after the application of energy to the tissue. Various configurations of sensor mechanism 150 are detailed below (see FIGS. 8A-8D).

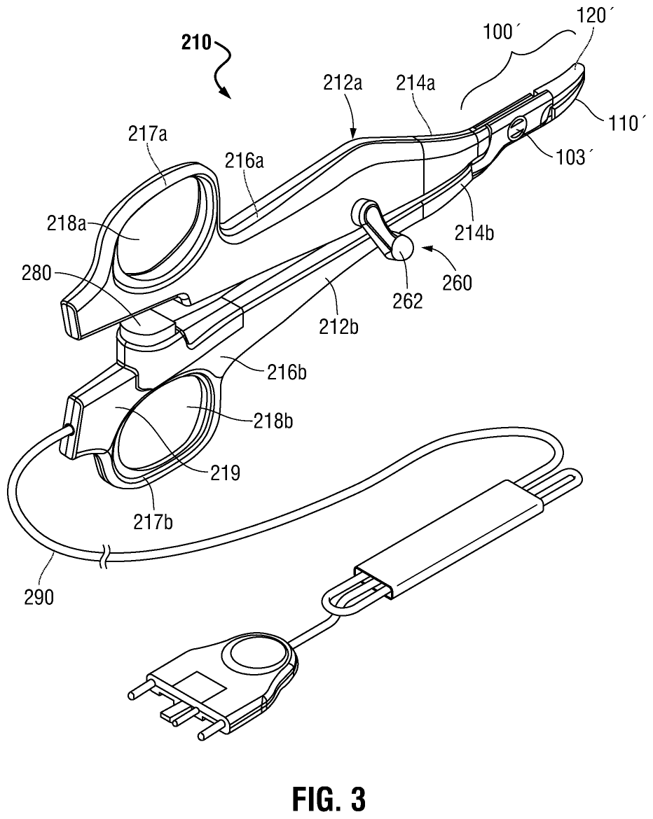

Referring to FIG. 3, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIGS. 2A and 2B). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to electrosurgical generator 400 (FIG. 1). Proximal shaft connector 219 secures a cable 290 to forceps 210 such that the user may selectively supply energy to jaw members 110′, 120′ for sealing tissue. More specifically, an activation switch 280 is provided for supplying energy to jaw members 110′, 120′ to seal tissue upon sufficient approximation of shaft members 212a, 212b, e.g., upon activation of activation switch 280 via shaft member 212a.

Forceps 210 further includes a trigger assembly 260 including a trigger 262 coupled to one of the shaft members, e.g., shaft member 212a, and movable relative thereto between an un-actuated position and an actuated position. Trigger 262 is operably coupled to a knife (not shown; similar to knife 64 (FIG. 2A) of forceps 10 (FIG. 1)) so as to actuate the knife to cut tissue grasped between jaw members 110,′ 120′ of end effector assembly 100′ upon movement of trigger 262 to the actuated position. Similarly as noted above with respect to forceps 10 (FIG. 1), other suitable cutting mechanisms are also contemplated.

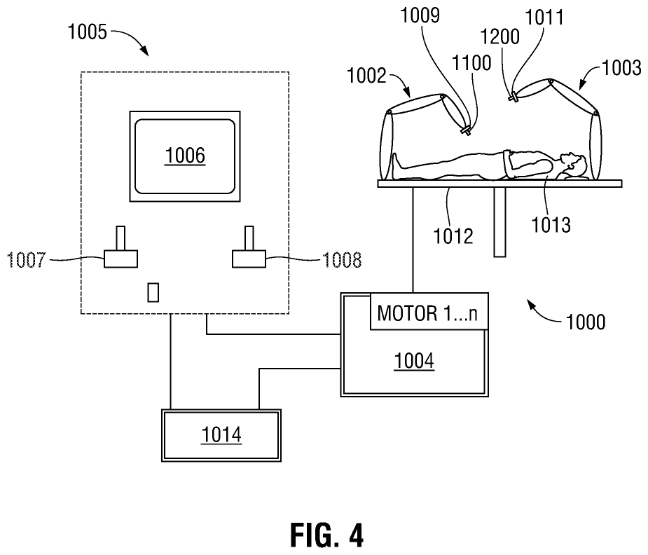

Referring to FIG. 4, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIGS. 2A and 2B), although other suitable end effector assemblies for coupling to attaching device 1009 are also contemplated. End effector assembly 1100 is connected to electrosurgical generator 400 (FIG. 1), which may be integrated into or separate from robotic surgical instrument 1000. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

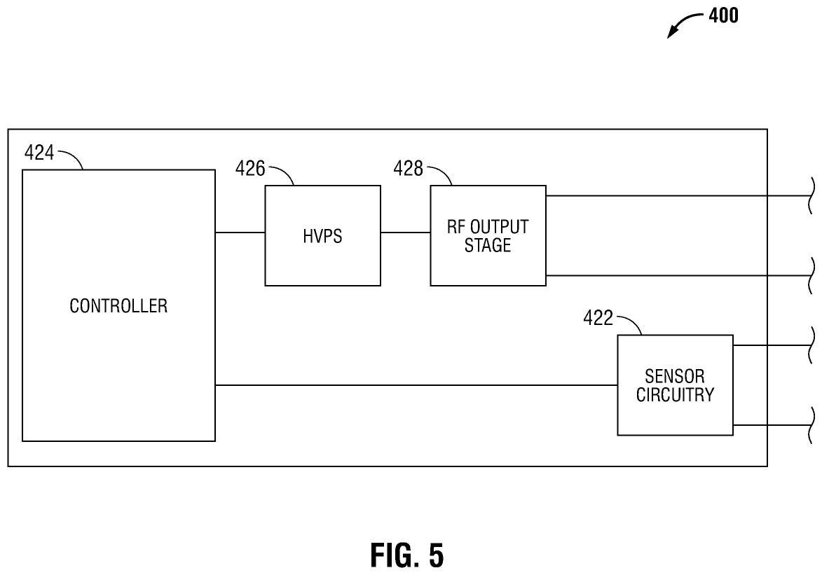

Referring to FIG. 5, electrosurgical generator 400 is shown as a schematic block diagram. Generator 400 may be utilized as a stand-alone generator (as shown in FIG. 1), may be incorporated into a surgical instrument 10, 210, 1000 (FIGS. 1, 3, and 4, respectively), or may be provided in any other suitable manner. Generator 400 includes sensor circuitry 422, a controller 424, a high voltage DC power supply (“HVPS”) 426 and an RF output stage 428. Sensor circuitry 422 is configured to receive sensor feedback from sensor mechanism 150 (FIGS. 2A and 2B), e.g., the one or more properties (mechanical, optical, chemical, electrical, etc.) of tissue grasped between jaw members 110, 120 (FIGS. 2A and 2B), and relay the same to controller 424. Controller 424 is configured to control the output of energy from HVPS 426 to RF output stage 428 and, thus, the application of energy from tissue-contacting surfaces 114, 124 of jaw members 110, 120 to tissue grasped therebetween (FIGS. 2A and 2B). More specifically, controller 424 is configured to receive the sensor feedback from sensor circuitry 422; determine, in real-time, a state, property, and/or other condition of the collagen based thereon; start, continue, modify, stop, etc., the output of energy from HVPS 426 to RF output stage 428 in order to facilitate sealing of tissue grasped between tissue-contacting surfaces 114, 124 of jaw members 110, 120 (FIGS. 2A and 2B); and/or determine, in real-time, if the tissue has been sufficiently sealed. Controller 424 is detailed below.

HVPS 426, under the direction of controller 424, provides high voltage DC power to RF output stage 428 which converts the high voltage DC power into RF energy for delivery to tissue-contacting 114, 124 of jaw members 110, 120, respectively, of end effector assembly 100 (see FIGS. 2A and 2B). In particular, RF output stage 428 generates sinusoidal waveforms of high frequency RF energy. RF output stage 428 may be configured to generate waveforms having various duty cycles, peak voltages, crest factors, and other parameters. Other suitable configurations are also contemplated such as for example, pulsed energy output, other waveforms, etc.

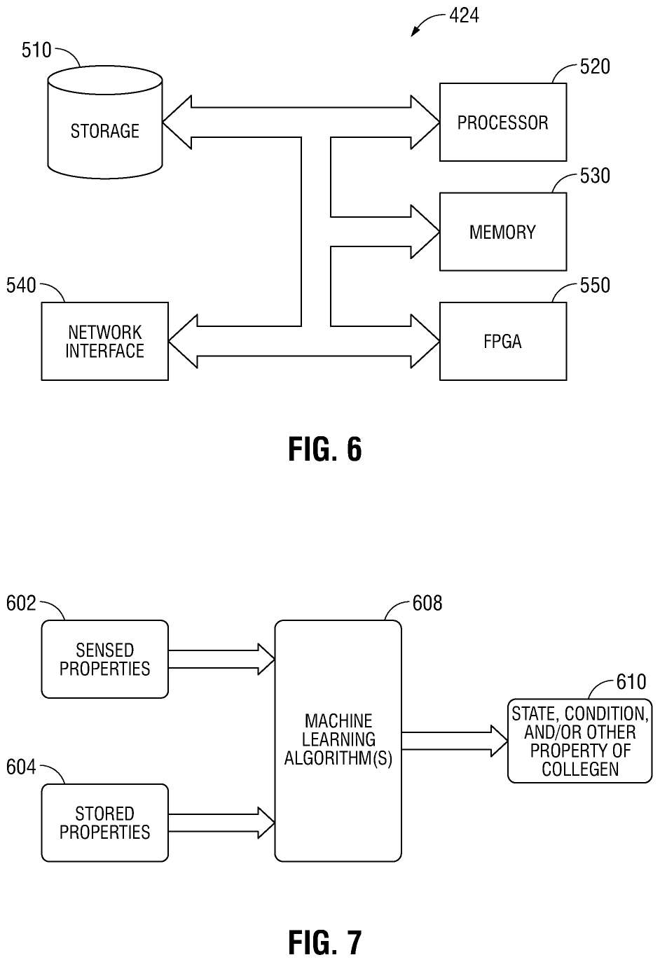

With additional reference to FIG. 6, controller 424 is configured to receive, from sensor circuitry 422, the one or more properties (mechanical, optical, electrical, etc.) of tissue grasped between jaw members 110, 120 (as sensed by sensor mechanism 150 (FIGS. 2A and 2B)) and, based thereon, determine, in real-time, a state, property, and/or other condition of the collagen in tissue before, during, and/or after the application of energy to the tissue. With respect to determining the state, property, and/or other condition of the collagen in tissue, this may be accomplished using, for example, a look-up table correlating the sensed property(s) to the state, property, and/or other condition of the collagen in tissue; a fixed algorithm determining the state, property, and/or other condition of the collagen in tissue based upon the sensed property(s); or a machine learning algorithm determining the state, property, and/or other condition of the collagen based upon the sensed property(s).

Referring particularly to FIG. 6, controller 424 includes a processor 520 connected to a computer-readable storage medium or a memory 530 which may be a volatile type memory, e.g., RAM, or a non-volatile type memory, e.g., flash media, disk media, etc. In embodiments, processor 520 may be, without limitation, a digital signal processor, a microprocessor, an ASIC, a graphics processing unit (GPU), field-programmable gate array (FPGA), or a central processing unit (CPU). In embodiments, memory 530 can be random access memory, read-only memory, magnetic disk memory, solid state memory, optical disc memory, and/or another type of memory. In embodiments, memory 530 can be separate from controller 424 and can communicate with processor 520 through communication buses of a circuit board and/or through communication cables such as serial ATA cables or other types of cables. Memory 530 includes computer-readable instructions that are executable by processor 520 to operate controller 424. In embodiments, controller 424 includes a network interface 540 to communicate with other computers or a server. In embodiments, a storage device 510 may be used for storing data. In embodiments, controller 424 may include one or more FPGAs 550. FPGA 550 may be used for executing various algorithms, e.g., fixed algorithms, machine learning algorithms, etc.

Memory 530 stores suitable instructions, to be executed by processor 520, for receiving the sensed data, e.g., sensed data from sensor circuitry 422 (FIG. 5), accessing storage device 510 of controller 424, and determining the state, property, and/or other condition of the collagen of tissue grasped between jaw members 110, 120 (FIGS. 2A and 2B) based upon the sensed data and information stored in storage device 510. Memory 530 further stores suitable instructions, to be executed by processor 520, to provide feedback based upon the determined state, property, and/or other condition of the collagen of tissue grasped between jaw members 110, 120 (FIGS. 2A and 2B). Although illustrated as part of generator 400, it is also contemplated that controller 424 be remote from generator 400, e.g., on a remote server, and accessible by generator 400 via a wired or wireless connection. In embodiments where controller 424 is remote, it is contemplated that controller 424 may be accessible by and connected to multiple generators 400.

With reference to FIGS. 6 and 7, in embodiments where one or more machine learning machine learning algorithms 608 are used, storage device 510 of controller 424 stores the one or more machine learning algorithms 608. The machine learning algorithm(s) 608 may be trained on and learn from stored settings 604, e.g., experimental data and/or data from previous procedures initially input into the one or more machine learning applications, in order to enable the machine learning application(s) to determine the state, property, and/or other condition of the collagen 610 based on the sensed property(s) 602. In embodiments, training the machine learning algorithm may be performed by a computing device outside of generator 400 and the resulting algorithm may be communicated to controller 424 of generator 400.

In embodiments, controller 424 receives the determined state, property, and/or other condition of the collagen 610 that was output from the machine learning algorithm 608 and communicates the same to a computing device, e.g., of controller 424, for use in controlling the output of energy from HVPS 426 to RF output stage 428. As noted above, this controlling may include starting, continuing, modifying, or stopping the output of energy. More specifically, a tissue sealing algorithm stored in storage device 510 of controller 424 may be implemented, modified, stopped, switched to another tissue sealing algorithm, etc.; the waveform output may modified, stopped, switched to another tissue sealing waveform; a setting may be changed, e.g., power may be increased or decreased; and/or an energy output time may be increased or decreased. That is, the energy output is adapted, if necessary, in accordance with the state, property, and/or other condition of the collagen determined. In this manner, the chemical and mechanical properties that define the tissue sealing process can be monitored and controlled to ensure that a sufficient tissue seal is achieved and, after formation, to check that a sufficient tissue seal was indeed created.

As one example, if it is determined that the collagen has not sufficiently denatured, liquefied, and/or crosslinked, the energy output may be adapted, as necessary, in order to ensure that denaturing, liquefying, and crosslinking occur to complete the tissue seal. On the other hand, where the collagen has denatured, liquefied, and crosslinked, the energy output may be stopped to avoid “overcooking” the tissue. Confirming the crosslinked collagen formation indicates that a sufficient tissue seal was created.

Controlling the energy output based upon the state, property, and/or other condition of the collagen is advantageous in that such control is directly based on the mechanical and chemical processes defining tissue sealing, that is, the denaturing, liquefying, and/or crosslinking of the collagen in tissue. This is in contrast to controls based on properties indicative of but not directly based upon the tissue sealing process itself, e.g., tissue impedance, temperature, hydration, compressibility, etc. It is noted that using, for example, one or more machine learning algorithms to determine the state, property, and/or other condition of the collagen, despite using indirect measurement inputs such as, for example, power, tissue impedance, tissue temperature, mechanical properties, chemical properties, etc., still enables control directly based on the tissue sealing process itself because such machine learning algorithm(s) are not controlling based upon these indirect measurement input but, instead, are controlling based on the determined state, property, and/or other condition of the collagen.

In some embodiments, the energy output may additionally or alternatively be controlled based upon tissue hydration. For example, water content at the beginning, middle, and/or end of collagen denaturing may be sensed (directly or indirectly), e.g., using a hydration sensor, and utilized in controlling the energy output. Tissue hydration may be useful because it has been found that as a result of collagen denaturing, water is unbound from the collagen molecules and thus becomes “free,” changing the tissue hydration.

Turning to FIGS. 8A-8D, various embodiments of sensor mechanisms 150 associated within jaw member 110 and/or jaw member 120 of end effector assembly 100 are detailed. As noted above, sensor mechanisms 150 are configured to communicate sensor feedback to sensor circuitry 422 of generator 400 (FIG. 5).

Referring initially to FIG. 8A, in embodiments, sensor mechanism 150 may include first and second leads 714, 724 connected (directly or indirectly) to tissue-contacting surfaces 114, 124 of jaw members 110, 120, respectively, or other electrodes associated with the end effector assembly 100 to sense electrical properties associated with the delivery of energy tissue to tissue “T” grasped between tissue-contacting surfaces 114, 124 such as, for example, current, voltage, power, impedance, slopes of these properties, etc. These electrical properties may be used in conjunction with one or more machine learning algorithms to determine the state, property, and/or other condition of the collagen of the tissue “T.”

As illustrated in FIG. 8B, sensor mechanism 150 may alternatively or additionally include an optical sensor assembly 730 including one or more optical transmitters 732 and one or more optical receivers 734 configured to cooperate to sense one or more optical properties of tissue “T” and provide the same to sensor circuitry 422. Although illustrated as positioned within knife channels 116, 126 defined within the first and second jaw members 110, 120, the one or more optical transmitters 732 and one or more optical receivers 734 may be disposed at any other suitable position on or within jaw member 110 and/or jaw member 120. Optical sensor assembly 730 may utilize fluorescence spectroscopy or other suitable optical measurement technique. These optical properties may indicate state, property, and/or other condition of the collagen of the tissue “T” or may be used in conjunction with one or more machine learning algorithms to determine the state, property, and/or other condition of the collagen of the tissue “T”.

With reference to FIG. 8C, sensor mechanism 150 may alternatively or additionally include one or more tissue-surface sensors 740 configured to contact a surface of tissue “T” grasped between jaw members 110, 120. Tissue-surface sensors 740 may be electrical sensors configured to sense electrical properties of tissue in contact therewith (e.g., impedance), mechanical property sensors configured to sense mechanical properties of tissue in contact therewith (e.g., texture, compressibility, etc.), temperature sensors configured to sense the temperature of tissue in contact therewith, chemical sensors configured to sense chemical properties of tissue in contact therewith, and/or other suitable sensors. The properties sensed may indicate the state, property, and/or other condition of the collagen of the tissue “T” or may be used in conjunction with one or more machine learning algorithms to determine the state, property, and/or other condition of the collagen of the tissue “T.”

Referring to FIG. 8D, sensor mechanism 150 may alternatively or additionally include one or more tissue-penetrating sensors 750 configured to penetrate tissue “T” grasped between jaw members 110, 120. Tissue-penetrating sensors 750 may be electrical sensors configured to sense electrical properties of the penetrated tissue (e.g., impedance), mechanical property sensors configured to sense mechanical properties of the penetrated tissue (e.g., texture, compressibility, etc.), temperature sensors configured to sense the temperature of the penetrated tissue, chemical sensors configured to sense chemical properties of the penetrated tissue, and/or other suitable sensors. The properties sensed may indicate the state, property, and/or other condition of the collagen of the tissue “T” or may be used in conjunction with one or more machine learning algorithms to determine the state, property, and/or other condition of the collagen of the tissue “T.”

It should be understood that various aspects disclosed herein may be combined in different combinations than the combinations specifically presented hereinabove and in the accompanying drawings. In addition, while certain aspects of the present disclosure are described as being performed by a single module or unit for purposes of clarity, it should be understood that the techniques of this disclosure may be performed by a combination of units or modules associated with, for example, a surgical system.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical system, comprising:
an end effector assembly including first and second jaw members each defining an electrically-conductive tissue-contacting surface, at least one of the first or second jaw members movable relative to the other between a spaced-apart position and an approximated position for grasping tissue between the tissue-contacting surfaces thereof, at least one of the first or second jaw members including a sensor configured to sense at least one property associated with the grasped tissue;
an electrosurgical generator including a controller and an energy output, the energy output configured to supply electrosurgical energy to the tissue-contacting surface of at least one of the first or second jaw members for conduction through the grasped tissue to seal the grasped tissue, the controller configured to:
receive the at least one sensed property;
determine at least one condition of collagen within the grasped tissue based upon the at least one sensed property; and
control the energy output based upon the determined at least one condition of the collagen within the grasped tissue.

2. The electrosurgical system according to claim 1, wherein the at least one condition of the collagen includes: denaturation of the collagen, migration of fibers of the collagen, restructuring of the collagen, crosslinking of the collagen, a type of the crosslinking of the collagen, a phase of the collagen, or a phase-change of the collagen.

3. The electrosurgical system according to claim 2, wherein the sensor includes an electrical sensor and wherein the controller includes a machine learning algorithm configured to determine the at least one condition of the collagen based upon the at least one sensed property received from the electrical sensor.

4. The electrosurgical system according to claim 1, wherein the sensor includes at least one of: an optical sensor, an electrical sensor, a mechanical property sensor, or a chemical sensor.

5. The electrosurgical system according to claim 1, wherein the controller is further configured to determine whether the grasped tissue is sufficiently sealed based upon the determined at least one condition of the collagen.

6. The electrosurgical system according to claim 1, wherein controlling the energy output includes at least one of: starting, modifying, continuing, or stopping the energy supplied to the at least one tissue-contacting surface.

7. The electrosurgical system according to claim 1, wherein the controller includes a storage device storing a machine learning algorithm configured to determine the at least one condition of collagen based upon the at least one sensed property.

8. The electrosurgical system according to claim 1, further comprising:
a housing; and
a shaft extending distally from the housing, wherein the end effector assembly is disposed at a distal end portion of the shaft.

9. The electrosurgical system according to claim 8, further comprising a manual actuator coupled to the housing and configured to move the at least one of the first or second jaw members between the spaced-apart position and the approximated position.

10. The electrosurgical system according to claim 1, further comprising:
first and second shaft members pivotably coupled to one another about a pivot, wherein the end effector assembly extends distally from the pivot, and wherein the first and second shaft members are movable relative to one another to move the at least one of the first or second jaw members between the spaced-apart position and the approximated position.

11. The electrosurgical system according to claim 1, further comprising:
a robotic arm, wherein the end effector assembly extends distally from the robotic arm.

12. A method of sealing tissue, comprising:
grasping tissue between electrically-conductive tissue-contacting surfaces of first and second jaw members;
supplying electrosurgical energy to the tissue-contacting surface of at least one of the first or second jaw members for conduction through the grasped tissue;
sensing at least one property associated with the grasped tissue;
determine at least one condition of collagen within the grasped tissue based upon the at least one sensed property; and
controlling the supplying of electrosurgical energy based upon the determined at least one condition of the collagen within the grasped tissue.

13. The method according to claim 12, wherein the at least one condition of the collagen includes: denaturation of the collagen, migration of fibers of the collagen, restructuring of the collagen, crosslinking of the collagen, a type of the crosslinking of the collagen, a phase of the collagen, or a phase-change of the collagen.

14. The method according to claim 13, wherein the at least one property is sensed by an electrical sensor and wherein determining the at least one condition includes implementing a machine learning algorithm to determine the at least one condition based upon the at least one sensed property sensed by the electrical sensor.

15. The method according to claim 12, wherein the sensed at least one property is an optical property, an electrical property, a mechanical property, or a chemical property.

16. The method according to claim 12, further comprising determining whether the grasped tissue is sufficiently sealed based upon the determined at least one condition of the collagen.

17. The method according to claim 12, wherein controlling the supplying of electrosurgical includes at least one of: starting, modifying, continuing, or stopping the supply of energy.

18. The method according to claim 12, wherein determining the at least one condition of collagen includes running a machine learning algorithm to determine the at least one condition of collagen based upon the at least one sensed property.
